(19) United States
(12) Patent Application Publication
(10) Pub. No.: US 2019/0169663 A1
(43) Pub. Date: Jun. 6, 2019

(54) MATERIAL PRETREATEMENT METHOD USING LOW-TEMPERATURE EXTRUSION AND MULTIENZYME SYNERGISTIC DEGRADATION

(71) Applicant: ZIBOHUICHUANG BIOLOGICAL SCIENTIFIC TECHNOLOGY LIMITED COMPANY, Shandong (CN)

(72) Inventors: Dechao SHEN, Shandong (CN); Xunye SHEN, Heilongjiang (CN); Xunyu SHEN, Shandong (CN)

(21) Appl. No.: 16/092,903

(22) PCT Filed: Apr. 7, 2017
(86) PCT No.: PCT/CN2017/079681

§ 371 Date: Oct. 11, 2018

(30) Foreign Application Priority Data
Apr. 11, 2016 (CN) 201610219779.5

(51) Int. Cl. C12P 19/02; C12P 7/64; C12P 7/04; C12P 19/22; C12P 21/06; C12P 19/14

(52) U.S. Cl. CPC C12P 19/02; C12P 7/64; C12P 7/04; C12P 2201/00; C12P 21/06; C12P 19/14; C12P 19/22

(57) ABSTRACT

A material pretreatment method using low-temperature extrusion and multienzyme synergistic degradation. A to-be-extruded material added with enzyme preparations and having a certain moisture content is blended before extrusion, the to-be-extruded material is fed into a screw extrusion device and is to be subjected to low-temperature extrusion treatment, multiple enzyme preparations are added in the material before extrusion, and starch, cellulose, protein and other substances in the material are degraded under the synergistic effect of the added multiple enzyme preparations in an extrusion process. Compared with the prior art, the yield of glucose and maltose converted from starch is improved, the residual oil rate of oil material meals is reduced, soluble substances of plant fiber materials are increased, and compared with the process of the traditional technology, the time needed in converting a protein material into a polypeptide material is shortened.

### CROSS-REFERENCE TO RELATED APPLICATION

This is the U.S. national stage of application for International Application No. PCT/CN2017/079681, filed on Apr. 7, 2017. Priority under 35 U.S.C. § 119(a) and 35 U.S.C. § 365(b) is claimed from Chinese Application No. 201610219779.5, filed on Apr. 11, 2016, the disclosures all of which are also incorporated herein by reference.

### TECHNICAL FIELD

The invention belongs to the technical field of pretreatment of materials, and the materials include: starch materials, oil materials, and vegetable fiber materials. The invention also relates to a pretreatment technique for proteinaceous materials.

### BACKGROUND ART

1. The Extrusion-Enzymolysis Studies of Starchy Materials

A large number of experimental studies conducted by domestic and foreign scholars have shown that starchy cereals and the starch thereof treated by extrusion with the addition of enzyme preparations have the starch degradation rates significantly faster than those of starchy cereals and the starch thereof treated without the addition of enzyme preparations.

The extrusion-enzymolysis studies conducted by foreign scholars are all limited to the study of gelatinization, liquefaction and saccharification of the extrudates of starchy cereals and the starch thereof treated by extrusion with the addition of only one enzyme preparation. The representative results are as follows: P. Linko and colleagues used a twin-screw extruder Clextral BC 45 to extrude the starches of barley and wheat added with Termamyl α-amylase. After extrusion, saccharification was carried out for 22 h, and the DE value was 97%. Linko et. al. studied the production of alcohol by the starch of cereals treated by extrusion with the addition of enzyme preparations (P. Linko et. al., 1989). Govindasamy S. et. al. used a twin-screw extruder to extrude sago starch added with thermostable α-amylase (Termamyl), and the extruded sago starch was liquefied at a high temperature, and then saccharified for 8 h to obtain a DE value of 83%-98% (S. Govindasamy, 0. H. Campanella, C. G. Oates, 1995). Vasanthan et. al. studied the dextrinization of starch in barley flour treated by extrusion with the addition of thermostable α-amylase (Termamyl) (Vasanthan et. al., 2001). Tim Baks et. al. studied the gelatinisation of wheat starch in a twin-screw extruder and the reaction between an enzyme and gelatinised starch mixtures (Tim Baks, Frans H. J. Kappen, Anja E. M. Janssen, Remko M. Boom, 2008). Normell Jhoe de Mesa-Stonestreet et. al. studied the production of sorghum protein concentrate by sorghum flour treated by extrusion with the addition of an enzyme preparation (Normell Jhoe de Mesa-Stonestreet, Sajid Alavi, Jeff Gwirtz, 2012).

The extrusion-enzymolysis studies conducted by domestic scholars are also limited to the study of the degradation of cereal starch treated by extrusion with the addition of only one enzyme preparation. For example, Ran Xu et. al. used a twin-screw extruder to study the regularity of converting corn starch treated by extrusion with the addition of a high-temperature-resistant α-amylase at an extrusion temperature (barrel temperature) of 85° C.-105° C. into reducing sugar (Ran Xu, Li Lingling, Jiang Linmao, Liu Xuewen, Wang Wenxian, 2006). Sun Yuqing and Ran Xu studied the feasibility of using a twin-screw extruder to extrude the corn starch added with only α-amylase at an extrusion temperature (barrel temperature) of 90° C.-100° C. to produce maltodextrin, focusing on the influences of three parameters of barrel temperature, material moisture content and enzyme concentration on the DE value of maltodextrin (Sun Yuqing, Ran Xu, 2006). Chinese scholars Hongyan Li et. al. applied the extrusion-enzymolysis technology to study the liquefaction, saccharification and fermentation processes for preparing rice wine using sticky rice added with only thermostable α-amylase at an extrusion temperature (barrel temperature) of 90° C.-110° C. (Hongyan Li, Zhengyu Jin*, Xueming Xu, 2013). Enbo Xu et. al. applied the extrusion-enzymolysis technology (with the addition of only thermostable α-amylase at an extrusion temperature (barrel temperature) of 98° C.-140° C.) to study the effects of extrusion-enzymolysis parameters on phenolics and antioxidant activity of glutinous rice (Enbo Xu, Zhengzong Wu, Jie Long, Fang Wang, Xueming Xu, Zhengyu Jin, Aiquan Jiao, 2015).

The Chinese patent No. ZL200710015601.X granted on Jun. 15, 2011 (invention title: Processing method, device and saccharification method for starch syrup raw material by extruding and adding enzyme) provides a processing method, a processing apparatus and a saccharification method of starch syrup raw materials added with enzyme preparations by extrusion cooking in a starch syrup production process. It is used to solve the problem that longer saccharification time is required for obtaining the syrup having a higher DE value (for example, DE=95%) in the prior art.

The Chinese patent No. ZL200710015601.X specifically discloses “the above-mentioned processing method is used for preparing a starch syrup raw material added with enzyme preparations by extrusion cooking, wherein the starch syrup raw material, before extrusion, is added with an appropriate amount of enzyme preparation including one or more of enzyme preparations like high-temperature-resistant α-amylase . . . mesophilic α-amylase, amyloglucosidase . . . complex enzyme.”

However, in fact, when the extrusion-enzymolysis technology studies were carried out by domestic and foreign scholars at present, the extrusion temperature is 85° C.-155° C. or ≤70° C.; only one enzyme preparation is added, including: thermostable α-amylase (Termamyl) or high-temperature-resistant α-amylase or mesophilic α-amylase. As a result, the degradation effect of starch in the extrusion-enzymolysis material has limitations, for example, the thermostable α-amylase (Termamyl) or the high-temperature-resistant α-amylase or the mesophilic α-amylase which can only degrade α-1,4 glycosidic bond cannot degrade α-1,6 glycosidic bond.

Table 1 is the verification results of the test of preparing glucose syrup by the extrudate from the low-temperature extrusion of the degermed corn flour added with only the high-temperature-resistant α-amylase in replace of corn starch in the Chinese patent No. ZL200710015601.X. The saccharification time is 12 h.

TABLE 1
Verification results of test of preparing glucose syrup by extrudate from the
low-temperature extrusion enzymolysis of dry-process degermed corn (April 2010)

| | | | | | | | | | | | resistant |
|---|---|---|---|---|---|---|---|---|---|---|---|
| | Filtration | | | Specific | | Starch | | | | Residual | starch |
| | rate/ | | DE | gravity/ | Syrup | conversion | Glucose | light | | starch | content of |
| No. | L(m² · h) | Solids/% | value/% | g/ml | yield/% | rate/% | content/% | transmittance/% | chromaticity | content/% | filter cake/% |
| 1 | 582.38 | 32.57 | 95.79 | 1.1264 | 103.69 | 95.64 | 27.29 | 92.78 | 1.20 | 0.85 | 0.45 |
| 2 | 726.67 | 33.12 | 98.71 | 1.1358 | 1085 | 98.79 | 29.98 | 96.59 | 1.00 | 0.45 | 0.26 |
| 3 | 686.23 | 32.88 | 96.66 | 1.1350 | 108.31 | 98.47 | 29.82 | 95.73 | 1.20 | 0.57 | 0.36 |

According to the verification results of the test of preparing the glucose syrup by the extrudate from the low-temperature extrusion of degermed corn flour added with only the high-temperature-resistant α-amylase in replace of corn starch in the Chinese patent No. ZL200710015601.X (date: Feb. 8 to 10, 2015, location: Chi Ping), the saccharification time is 15 h, the DE value=98.78%, and the DX value=96.78%.

The Chinese patent No. ZL201310300202.3 granted on Jun. 3, 2015 (invention title: A saccharifying method for producing maltose syrup through extruded syrup raw materials with the addition of enzymes) relates to a saccharification process for the extrudate of the extruded syrup raw material added with enzyme preparations for producing maltose syrup, where syrup mixing and high-temperature (128° C.-145° C.) enzyme inactivation in the prior art are omitted, and the extruded syrup raw materials with the addition of enzyme preparations are liquefied and saccharified directly.

However, the Chinese Patent No. ZL201310300202.3 does not mention the enzyme preparations added to the extruded syrup raw materials with the addition of enzyme preparations for producing maltose syrup. As described above, as to the researches with the extrusion-enzymolysis technology carried out by domestic and foreign scholars at present, only one enzyme preparation is added.

In fact, the Chinese patent No. ZL201210421665.0 (invention title: An extrusion enzymolysis and saccharification method for producing ultrahigh maltose syrup raw material) granted on Nov. 5, 2014 relates to an extrusion enzymolysis and saccharification method for producing ultrahigh maltose syrup raw material. Its main technical features are providing an extrusion processing method of a maltose syrup raw material added with enzyme preparation for producing ultrahigh maltose syrup, and a saccharification method using the extrudate of a maltose syrup raw material added with enzyme preparation. Compared with the conventional multi-enzyme synergistic method for producing maltose syrup without the extrusion of the maltose syrup raw materials as commonly used at present, the saccharification time required to achieve a maltose content of more than 90% in the saccharification liquid obtained without the extrusion of the enzyme-added maltose syrup raw materials as in the current conventional practice is only ⅔ to ½ of the current conventional saccharification time, and the starch production process and jet liquefaction process are omitted to avoid the environmental pollution caused by starch production. The invention can be used for producing an ultrahigh maltose syrup having a maltose content of more than 90%, and can also be used for producing a common maltose syrup having a maltose content of less than 60% and a high maltose syrup having a maltose content of 60%-80%.

However, as mentioned above, at present, domestic and foreign scholars only added one enzyme preparation in their studies of the extrusion-enzymolysis technology of maltose syrup raw materials (see Cai Yuling, Experimental Study on Degermed Corn Added Amylase Extruded at Low Temperature for Production Ultra-high Maltose Syrup [D]. Shandong Zibo: Shandong University of Technology, May 2011; Jin Zhengyu, A method for producing maltose syrup and oligo-isomaltose by extrusion added with enzyme and liquefaction (P). Application No.: CN201610222609.2, Jiangnan University, Apr. 11, 2016), wherein Cai Yuling added β-amylase preparation to the materials before extrusion to prepare the extrudate for producing maltose syrup; Jin Zhengyu added high-temperature-resistant α-amylase to prepare the extrudate for producing maltose syrup).

From 2004 to 2014, my students and I conducted the degradation process in extruder of starch in rice and corn added with only one enzyme preparation (amylase, such as high-temperature-resistant α-amylase preparation or mesophilic α-amylase preparation or β-amylase preparation) by low temperature extrusion at an extrusion temperature ≤80° C. The results of a large number of laboratory researches and production pilot tests show that we have completed the following research contents:

(1) The mechanical degradation, enzymatic degradation process in the extruder of starch in rice and corn added with only one enzyme preparation improves the saccharification effect of the extrudate; the inactivation regularity of the enzyme preparation added during the extrusion process along the screw length (Ma Chengye, 2010; Chen Shanfeng, 2012; Cai Yuling, 2011);

(2) Revealing the regularity of forming substances from the interaction of the two or three of starch, protein and lipid of rice and degermed corn added with only one enzyme preparation in the extruder during the low temperature extrusion process (extrusion temperature ≤70° C.) and its influence on saccharification index (Ma Chengye, 2010; Chen Shanfeng, 2012);

(3) Studying the regularity of the influence of low-temperature-extrusion system parameters on the main inspection indices of the subsequent gelatinization, liquefaction and saccharification processes of extrudates added with only one enzyme preparation (Shen Dechao et. al., 2007; Xi Kewei, 2007; Ji Weiguang, 2009; Zhang Chunye, 2008; Ma Chengye, 2010; Cai Yuling, 2011; Chen Shanfeng, 2012; Shen Xunyu et. al., 2014).

The deficiency of the above-mentioned researches carried out by domestic and foreign scholars is that since α-amylase is an endo-amylase, it can randomly hydrolyze the α-1,4 glycosidic bonds in starch, soluble dextrin and oligosaccharides, and the hydrolysis would form dextrin and small amounts of glucose and maltose (Jiang Xirui, Duan Gang, 2003). Usually, cereal starch is composed of amylose and amylopectin. Amylose is mainly composed of glucoses linked by α-1,4 glycosidic bonds. Amylopectin contains α-1,4 glycosidic bonds and α-1,6 glycosidic bonds. At present, domestic and foreign scholars are limited to adding only high-temperature-resistant α-amylase or mesophilic α-amylase or β-amylase preparations which cannot degrade α-1,6 glycosidic bonds of amylopectin, when studying the extrusion-enzymolysis technology of cereals and starches.

From 2003 to 2014, we conducted the experimental research on the starch in dry-process degermed corn using the low-temperature extrusion enzymolysis, which was also limited to the experimental research on the extrusion of dry-process degermed corn added with only high-temperature-resistant α-amylase in replace of corn starch to produce syrup. The results showed that the saccharification carried out for 12 h achieved the syrup DE value of 95% (DX value is about 93%) (Shen Xunyu, 2014); the saccharification carried out for 15 h achieved the syrup DE value of 98.875% (DX value=96.875%).

From October of 2015 to the present, our preliminary studies on the low-temperature extrusion-multi-enzyme synergistic degradation of starch in corn showed that the use of the extrudate from low-temperature extrusion of the degermed corn added with a number of enzyme preparations (for example, the addition of high-temperature-resistant α-amylase, glucoamylase and pullulanase into the materials to be extruded before extrusion) as a syrup raw material (extrusion temperature ≤80° C.) to produce glucose syrup required the saccharification time of 15 h and achieved a DE value of about 102%. The syrup sample was tested by Shandong Institute for Product Quality Inspection. The glucose content of the above glucose syrup was 27.5% (Shandong Product Quality Inspection Research Institute. Test Report of Starch Syrup, 2016). According to our tests, the solids content of syrup was 25.5% and 25.25% and the average value was 25.375, so the DX value of syrup=(27.5/(25.375×1.1015))×100%=98.39%, wherein 1.1015 was the relative density of syrup (our test results).

Table 2 is the results of liquid chromatography analysis of the preparation of glucose syrup using the extrudate from the low-temperature extrusion of dry-process degermed corn added with a number of enzyme preparations (e.g., high-temperature-resistant α-amylase, glucoamylase and pullulanase) in replace of starch conducted from Apr. 27, 2016 to May 11, 2016.

TABLE 2
Liquid chromatography analysis results of preparation
of glucose syrup by the extrudate from low-temperature
extrusion enzymolysis of dry-process degermed corn

| | percent % name | | | | |
|---|---|---|---|---|---|
| | oligosac- | malt | malto- | malto- | glu- |
| date | charide/% | glucan/% | triose/% | biose/% | cose/% |
| 2016 May 11 | 0.18 | 0.16 | 0.26 | 2.4 | 97.00 |
| 2016 May 11 | 0.12 | 0.13 | 0.26 | 2.41 | 97.08 |
| 2016 Apr. 27 | 0.17 | 0.37 | 0.41 | 2.43 | 96.67 |
| 2016 Apr. 27 | 0.13 | 0.35 | 0.43 | 2.89 | 96.19 |

At present, in the saccharification process using corn starch as raw material to produce syrup, the saccharification liquid requires saccharification for 50-60 h, and the syrup has a glucose content of 96.67%. Table 2 is the liquid chromatography analysis results of the preparation of glucose syrup by dry-process degermed corn extrudate in replace of starch using the low-temperature extrusion multi-enzyme synergistic degradation of the present invention, and shows that starch is converted into glucose with the DX value up to 97.07% after saccharification of 13 h-15 h. (see Table 2, date: May 11, 2016, location: Shandong Rizhao).

It can be seen that low-temperature extrusion-multi-enzyme synergistic degradation of starch in the extrudate of the degermed corn added with a number of enzyme preparations can overcome the limitation of the extrusion with the addition of only one enzyme preparation (such as high-temperature-resistant α-amylase) which can only degrade α-1,4 glycosidic bond in starch, but cannot degrade α-1,6 glycosidic bond of starch in the degermed corn, and can increase the glucose content in syrup (see Tables 1 and 2).

2. Extrusion-Enzymolysis Studies of Proteinaceous Materials

At present, the processes for preparing proteins and polypeptides are generally as follows: preparation of raw materials for proteins and polypeptides→pretreatment (including: pulverization, ultrafine pulverization, microwave treatment, ultrasonic treatment, extrusion treatment (Note: the material to be extruded is not added with any chemical reagents and enzyme preparations before extrusion), etc.)→separation (single enzyme and multi-enzyme synergistic hydrolysis or alkali solution and acid isolation)→purification→ultrafiltration→concentration→spray drying→production into powdered protein or polypeptide products (Shi Yanguo, Ren Li, Soybean Product Technology [M]. Beijing: China Light Industry Press. 1998.3; Huang Yuyang, Li Yang, Li Yongping, Using the Response Surface Method to Optimize the Corn Gluten Technique which the Untrasonic Help Assist Biological Active Peptide {J}, Grain Processing, 2010, 35(3):56-60; Liu Jing, Zhang Guanghua, Microwave heating to hydrolysis soybean protein into low molecular weight peptides by enzymes' cooperation {J}, Food Science and Technology, 2010, 35(6): 39-43).

The above-mentioned processes for preparing proteins and polypeptides have longer process time, lower protein extraction rates, lower conversion rates of protein to polypeptide, and higher production cost.

The Chinese patent No. ZL201210396360.9 (invention title: Preparation of protein and polypeptide through low-temperature extrusion enzymolysis method) granted on Nov. 5, 2014 provides a low-temperature extrusion enzymolysis method for the preparation of protein and polypeptide, including a low-temperature extrusion enzymolysis method of raw materials added with enzyme preparations for preparing protein and polypeptide, and a method of preparing a powdery protein or polypeptide product through carrying out conventional extraction→separation (single enzyme and multi-enzyme synergistic hydrolysis or alkali extraction and acid precipitation)→purification→ultrafiltration→concentration→spray drying or freeze drying on pulverized substances obtained after low-temperature drying of extrudates of raw materials added with enzyme preparations for preparing protein and polypeptide. The Chinese Patent No. ZL201210396360.9 specifically discloses “the raw material added with enzyme preparations for preparing protein and polypeptide extruded by the low-temperature extrusion enzymolysis method of the raw material added with enzyme preparations for preparing protein and polypeptide, wherein the appropriate amount of enzyme preparations added before the extrusion of the raw material for preparing protein and polypeptide includes an appropriate amount of one or more of alkaline protease, neutral protease, acid protease, complex protease, and papain.”

However, according to the researches of my students and I (Wu Jing, Experimental study on extruded corn degermed with enzyme for production syrup and extracting protein from residue filtered (D), Shanxi Taigu: Shanxi Agricultural University. June 2010) and Examples 1 and 2 of the Chinese patent No. ZL201210396360.9, when corn polypeptide powder and soy isolate protein powder are prepared, only one enzyme preparation is added to the raw material for preparing protein and polypeptide before extrusion processing: neutral protease or alkaline protease, which limits protein degradation and the degree of conversion into polypeptide. It directly affects the time required for the process of preparing proteins and polypeptides, as well as the rate of protein extraction and the rate of converting proteins into polypeptides. In addition, research reports on the pretreatment of protein materials by a low-temperature extrusion-multi-enzyme synergistic degradation method have not been found.

3. With Regard to Oil Materials and Vegetable Fiber Materials

For the pretreatment method of oil materials and vegetable fiber materials, research reports on the pretreatment of oil materials and vegetable fiber materials by a low-temperature extrusion-multi-enzyme synergistic degradation method have not been found.

### MAIN REFERENCES

[1] C. Mercier, P. Linko, J. M. Harper. Extrusion Cooking. American Association of Cereal Chemists, Inc. St. Paul, Minn., USA, 1989: 235-245
[2] S. Govindasamy, 0. H. Campanella, C. G Oates. Influence of extrusion variables on subsequent saccharification behaviour of sago starch. Food Chemistry. 1995.54(3):289-296
[3] Thava Vasanthan, Judy Yeung, Ratnajothi Hoover. Dextrinization of starch in barley flours with thermostable alpha-amylase by extrusion cooking. Starch. 2001, 53(12): 616-622.
[4] Tim Baks, Frans H. J. Kappen, Anja E. M. Janssen. Remko M. Boom. Towards an optimal process for gelatinisation and hydrolysis of highly concentrated starch-water mixtures with alpha-amylase from B. licheniformis. Journal of Cereal Science. 2008, 47(2): 214-225.
[5] Normell Jhoe de Mesa-Stonestreet, Sajid Alavi, Jeff Gwirtz. Extrusion-enzyme liquefaction as a method for producing sorghum protein concentrates. Journal of Food Engineering 2012 (108): 365-375
[6] Hongyan Li, Zhengyu Jin, Xueming Xu, Design and optimization of an efficient enzymatic extrusion pretreatment for Chinese rice wine fermentation. Food Control 32 (2013) 563-568
[7] Enbo Xu, Zhengzong Wu, Jie Long, Fang Wang, Xueming Xu, Zhengyu Jin, Aiquan Jiao. Improved bio accessibility of phenolics and antioxidant activity of glutinous rice and its fermented Chinese rice wine by simultaneous extrusion and enzymatic hydrolysis. Journal of Functional Foods 17 (2015): 214-216
[8] Jiang Xirui, Duan Gang, Practical Technical Manual of Enzyme Preparation, China Light Industry Press, 2003: 21-30
[9] Ran Xu, Li Lingling, Jiang Linmao, Liu Xuewen, Wang Wenxian, Influence of Extrusion Conditions on Conversion Extent of Corn Amylase, Cereal and Feed Industry, 2006(7):23-25
[10] Sun Yuqing, Ran Xu, Research of Production of Maltodextrin by Extrusion, Food Science and Technology, 2006(10): 123-125, 137
[11] Shen Dechao, Xi Kewei, Ma Chengye, Zhang Shuang, Shen Xunyu, Experimental Study on Saccharification Process of Extrudate at Low Temperature for Corn Crude Starch Added Enzyme Preparation, Transactions of Chinese Society of Agricultural Engineering, 2007, 23(12):263-268
[12] Xi Kewei, The Study of Producing Glucose Syrup with Extruded Corn Starch and Degermed Maize, Dissertation for the Master Degree, [D]. Harbin: Northeast Agricultural University, 2007
[13]. Zhang Chunye, Study on extruded maize without germ added enzyme preparation for producing alcohol, Dissertation for the Master Degree, [D]. Harbin: Northeast Agricultural University, 2008
[14]. Ji Weiguang, The Study on Extruded Rice with Enzyme Added as Beer Adjunct, Dissertation for the Master Degree, [D]. Baoding: Agricultural University of Hebei, 2009
[15]. Cai Yuling, Experimental Study on Degermed Corn Added Amylase Extruded at Low Temperature for Production Ultra-high Maltose Syrup, Dissertation for the Master Degree, [D]. Zibo: Shandong University of Technology, 2011
[16] Ma Chengye, Saccharification Experiment Study on Degermed Corn Added with Amylase Extruded at Low Temperature for Production of Syrup: [PhD thesis] [D]. Harbin: Northeast Agricultural University, 2010
[17] Chen Shanfeng, The Experimental Study on Rice Added with Enzymes Extruded at Low Temperature Used as Beer Adjunct: [PhD thesis] [D]. Baoding: Agricultural University of Hebei, 2012
[18]. Test Report No. 050052, Shandong Product Quality Inspection Research Institute, 2016
[19]. You Xin, Production and Application of Starch Sugar Handbook, China Light Industry Press, 1999: 93, 144
[20]. Shen Xunyu, Shen Dechao, The technology of starch replaced by dry degermed maize extruded at low temperature and degraded by enzyme for syrup production, Journal of Shandong University of Technology, 2014, 28(6): 55-58
[21]. Shi Yanguo, Ren Li, Soybean Product Technology [M], Beijing: China Light Industry Press, 1998: 3
[22]. Huang Yuyang, Li Yang, Li Yongping, Using the Response Surface Method to Optimize the Corn Gluten Technique which the Untrasonic Help Assist Biological Active Peptide {J}, Grain Processing, 2010, 35(3):56-60
[23]. Liu Jing, Zhang Guanghua, Microwave heating to hydrolysis soybean protein into low molecular weight peptides by enzymes' cooperation {J}, Food Science and Technology, 2010, 35(6): 39-43
[24]. Xiao Zhigang, The Experiment of Extruded Maize without Germ to Produce Starch Syrup, Dissertation for the Master Degree, [D]. Harbin: Northeast Agricultural University, 2002
[25]. Xiao Zhigang, The Study of Production of High DE Value Syrup Used Extrusion of Corn without Germ and Application of Its Byproduct, [PhD thesis] [D]. Harbin: Northeast Agricultural University, 2005
[26]. Shen Dechao, Noticeable issues in the development of citric acid industry—there is an urgent need in increasing the starch conversion rate of raw corn, Shanhai: Collected Papers of Forum Series of China Biotech Fermentation Industry in 2015, China Fermentation Industry Association: 46-53
[27]. Shen Dechao, Xiao Zhigang, Empirical formulas relating to extrusion of degermed maize for production of starch syrup based on function theory of dimension analysis, Transactions of the CASE, 2005, 21(12): 25-29
[28]. Meng Yang, Mashing Experiment of Beer with Extruded Rice as Adjuncts, [PhD thesis] [D]. Harbin: Northeast Agricultural University, 2001
[29]. Jiang Yiyuan, Verification of the Theory for a Sphere Settling in Fluid with Resistance in Different Ranges by Experiment of i-Testing Design, Transactions of the CSAE, 1996, 12(3):12-15
[30]. Xiao Zhigang, Shen Dechao, Empirical formula relating to extrusion parameters and ratio of starch syrup based on dimensional analysis, Journal of Food Science and Technology, 2014, 32(4): 21-27
[31]. Meng Yang, Mashing Experiment of Beer with Extruded Rice as Adjuncts, [PhD thesis] [D]. Harbin: Northeast Agricultural University, 2001
[32] Chinese patent No. ZL200710015601. X (grant announcement date: Jun. 15, 2011)
[33] Chinese patent No. ZL201210396360.9 (grant announcement date: Nov. 5, 2014)
[34] Chinese patent No. ZL201310300202.3 (grant announcement date: Jun. 3, 2015)
[35] Chinese patent No. ZL201210421665.0 (grant announcement date: Nov. 5, 2014)
[36] Chinese patent Application No. CN201610222609.2 (publication (announcement) date: Jun. 15, 2016)
[37] Wu Jing, Experimental Study on Extruded Corn Degermed with Enzyme for Production Syrup and Extracting Protein from Residue Filtered (D). Shanxi Taigu: Shanxi Agricultural University. June 2010

### SUMMARY OF THE INVENTION

The present invention provides:

(1) a material pretreatment method by low-temperature extrusion-multi-enzyme synergistic degradation, including preparing the material to be extruded added with enzyme preparations and having a certain moisture content before extrusion, wherein a plurality of enzyme preparations are added to the material before extrusion, and then, the material to be extruded is fed into a screw extrusion device for low-temperature extrusion processing to form an extrudate;

(2) a material pretreatment method by low-temperature extrusion-multi-enzyme synergistic degradation, including preparing the material to be extruded added with enzyme preparations and having a certain moisture content before extrusion, wherein the added enzyme preparations include: one or more of added high-temperature-resistant α-amylase, highly-efficient thermostable α-amylase, mesophilic α-amylase, amyloglucosidase, glucoamylase, highly-efficient amyloglucosidase, potent amyloglucosidase, protease, complex enzyme, etc., and one or more of added alkaline protease, neutral protease, acid protease, complex protease and papain; the added enzyme preparations further include: one or more of added complex amyloglucosidase, pullulanase, maltobiohydrolase, maltotriohydrolase, cellulase, hemicellulase, pectinase, xylanase, β-amylase, fungal α-amylase, fuel alcohol protease, alcohol-specific complex amyloglucosidase, and then, the material to be extruded is fed into a screw extrusion device for low-temperature extrusion processing;

(3) a material pretreatment method by low-temperature extrusion-multi-enzyme synergistic degradation, including preparing the material to be extruded added with enzyme preparations and having a certain moisture content before extrusion, wherein the added enzyme preparations include: two or more of added high-temperature-resistant α-amylase, highly-efficient thermostable α-amylase, mesophilic α-amylase, amyloglucosidase, glucoamylase, highly-efficient amyloglucosidase, potent amyloglucosidase, protease, complex enzyme, etc., and two or more of added alkaline protease, neutral protease, acid protease, complex protease and papain; the added enzyme preparations further include: two or more of complex amyloglucosidase, pullulanase, maltobiohydrolase, maltotriohydrolase, cellulase, hemicellulase, pectinase, xylanase, β-amylase, fungal α-amylase, fuel alcohol protease, alcohol-specific complex amyloglucosidase, and then, the material to be extruded is fed into a screw extrusion device for low-temperature extrusion processing to form an extrudate;

(4) a material pretreatment method by low-temperature extrusion-multi-enzyme synergistic degradation according to (1), (2) or (3), including preparing the material to be extruded added with enzyme preparations and having a certain moisture content before extrusion, the material to be extruded including: one or more of rice, degermed corn, corn with germ, sorghum rice, barley, wheat and their starch, and crude starch added with enzyme preparations, and one or more of cassava whole flour, potato whole flour and their starch, and crude starch added with enzyme preparations, and the material to be extruded further including: one or more starchy materials of sweet potato whole flour, millet, black kerneled rice, buckwheat, oat, rye and their starch, and crude starch added with enzyme preparations;

(5) a material pretreatment method by low-temperature extrusion-multi-enzyme synergistic degradation according to (1), (2) or (3), including preparing the material to be extruded added with enzyme preparations and having a certain moisture content before extrusion, the material to be extruded including: one or more oily materials of soybean, corn germ, peanut, sunflower seed, rapeseed, sesame seed, yellow mustard seed, cottonseed, flax seed, castor seed, and perilla seed added with enzyme preparations;

(6) a material pretreatment method by low-temperature extrusion-multi-enzyme synergistic degradation according to (1), (2) or (3), including preparing the material to be extruded added with enzyme preparations and having a certain moisture content before extrusion, the material to be extruded including: stem, leaf, pod, root, fruit vegetable fiber materials from one or more of corn, wheat, barley, rice, sorghum, rape, soybean plants added with enzyme preparations;

(7) a material pretreatment method by low-temperature extrusion-multi-enzyme synergistic degradation according to (1), (2) or (3), including preparing the material to be extruded added with enzyme preparations and having a certain moisture content before extrusion, the material to be extruded including: one or more materials in the filtered mash residues from brewing beer with rice, degermed corn, sorghum rice, barley and wheat as auxiliary materials, one or more materials in the filtered residues of sugar liquid from producing syrup with rice, degermed corn, sorghum rice, barley and wheat as raw materials, one or more materials in the dregs or filtered residues from producing alcohol with rice, degermed corn, corn with germ, sorghum rice, barley and wheat as raw materials, one or more materials in the defatted cakes from producing oil with soy, corn germ, peanut, sunflower seed, rapeseed, sesame, yellow mustard, rice bran and cottonseed as raw materials, and one or more proteinaceous materials in soy protein powder, corn protein powder, barley protein powder, wheat protein powder, peanut protein powder, sunflower seed protein powder, rapeseed protein powder, rice bran protein powder, sesame protein powder and cottonseed protein powder;

(8) a material pretreatment method by low-temperature extrusion-multi-enzyme synergistic degradation according to (1), (2) or (3), including preparing the material to be extruded added with enzyme preparations and having a certain moisture content before extrusion, wherein the moisture content of the material to be extruded is ≤70%;

(9) a material pretreatment method by low-temperature extrusion-multi-enzyme synergistic degradation according to (1), (2) or (3), including preparing the material to be extruded added with enzyme preparations and having a certain moisture content before extrusion, and then, feeding the material to be extruded into a screw extrusion device for low-temperature extrusion processing, wherein the screw extrusion device comprises one or more of a single-screw extruder, a twin-screw extruder, and a tri-screw extruder;

(10) a material pretreatment method by low-temperature extrusion-multi-enzyme synergistic degradation according to (1), (2) or (3), including preparing the material to be extruded added with enzyme preparations and having a certain moisture content before extrusion, and then, feeding the material to be extruded into a screw extrusion device for low-temperature extrusion processing, wherein the screw extrusion device has the extrusion barrel temperature of ≤80° C.

The above contents of the present invention will be better understood by referring to the following detailed description and examples.

### DETAILED DESCRIPTION OF THE INVENTION

The present invention provides a material pretreatment method by low-temperature extrusion-multi-enzyme synergistic degradation, including preparing the material to be extruded added with enzyme preparations and having a certain moisture content before extrusion, wherein the extrusion barrel temperature is ≤80° C.; the moisture content of the material is ≤70%; a number of enzyme preparations are added into the material before extrusion, including: two or more of α-amylase, glucoamylase, maltase, pullulanase and protease. During the extrusion process of the material, the substances like starch, cellulose and protein in the material are degraded by the synergistic action of a variety of added enzyme preparations all the time.

Compared with the prior art, the present invention improves the yield of converting starch into glucose and maltose, reduces the meal residual oil rate after oil production by solvent extraction of oil materials, increases the soluble substances of vegetable fiber materials, and increases the protein extraction rate of proteinaceous materials. The above deficiencies of the prior art are solved.

The processing measures of the technology of the present invention are as follows:

Step 1: a material pretreatment method by low-temperature extrusion-multi-enzyme synergistic degradation, including preparing the material to be extruded added with enzyme preparations and having a certain moisture content before extrusion, wherein the added enzyme preparations include: one or more of added high-temperature-resistant α-amylase, highly-efficient thermostable α-amylase, mesophilic α-amylase, amyloglucosidase, glucoamylase, highly-efficient amyloglucosidase, potent amyloglucosidase, protease, complex enzyme, etc., and one or more of added alkaline protease, neutral protease, acid protease, complex protease and papain; the added enzyme preparations further include: one or more of added complex amyloglucosidase, pullulanase, maltobiohydrolase, maltotriohydrolase, cellulase, hemicellulase, pectinase, xylanase, β-amylase, fungal α-amylase, fuel alcohol protease, alcohol-specific complex amyloglucosidase, and then, the material to be extruded is fed into a screw extrusion device for low-temperature extrusion processing; the screw extrusion device comprises one or more of a single-screw extruder, a twin-screw extruder, and a tri-screw extruder; the screw extrusion device has the extrusion barrel temperature of ≤80° C.; the moisture content of the material to be extruded is ≤70% before extrusion;

Step 2: preparing the material to be extruded added with enzyme preparations and having a certain moisture content before extrusion, the material to be extruded including: one or more of rice, degermed corn, corn with germ, sorghum rice, barley, wheat and their starch, and crude starch added with enzyme preparations, and one or more of sweet potato whole flour, cassava whole flour, potato whole flour and their starch, and crude starch added with enzyme preparations, and the material to be extruded further including: one or more starchy materials of millet, black kerneled rice, buckwheat, oat, rye added with enzyme preparations;

preparing the material to be extruded added with enzyme preparations and having a certain moisture content before extrusion, the material to be extruded including: one or more oily materials of soybean, corn germ, peanut, sunflower seed, rapeseed, sesame seed, yellow mustard seed, cottonseed, flax seed, castor seed, and perilla seed added with enzyme preparations;

preparing the material to be extruded added with enzyme preparations and having a certain moisture content before extrusion, the material to be extruded including: stem, leaf, pod, root, fruit vegetable fiber materials from one or more of corn, wheat, barley, rice, sorghum, rape, soybean plants added with enzyme preparations;

preparing the material to be extruded added with enzyme preparations and having a certain moisture content before extrusion, the material to be extruded including: one or more materials in the filtered residues of beer mash with rice, degermed corn, sorghum rice, barley and wheat as auxiliary materials added with enzyme preparations, one or more materials in the filtered residues of sugar liquid with rice, degermed corn, sorghum rice, barley and wheat as raw materials for syrup, one or more materials in the dregs or filtered residues with rice, degermed corn, corn with germ, sorghum rice, barley and wheat as raw materials for alcohol, one or more materials in the defatted cakes with soy, corn germ, peanut, sunflower seed, rapeseed, sesame, yellow mustard, rice bran and cottonseed as raw materials for oil production, and one or more proteinaceous materials in soy protein powder, corn protein powder, barley protein powder, wheat protein powder, peanut protein powder, sunflower seed protein powder, rapeseed protein powder, rice bran protein powder, sesame protein powder and cottonseed protein powder.

In one embodiment, the present invention relates to low-temperature extrusion multi-enzyme synergistic degradation of starch in a degermed corn extrudate, and can overcome the limitation of the aforementioned prior art about adding only one enzyme preparation during extrusion. For example, as to starchy materials, adding high-temperature-resistant α-amylase can only degrade α-1,4 glycosidic bond of starch, but cannot degrade α-1,6 glycosidic bond of starch in degermed corn.

The technology of the present invention, low-temperature extrusion—multi-enzyme synergistic degradation of materials (such as starchy dry-process degermed corn), i.e., low-temperature extrusion of materials (such as starchy degermed corn) added with a number of enzyme preparations (such as high-temperature-resistant α-amylase, glucoamylase and pullulanase), can overcome the above-mentioned deficiencies of the prior art, and increase the glucose content and the maltose content in syrup.

In another embodiment, the present invention provides a method for the preparation of proteins and polypeptides by low-temperature extrusion-multi-enzyme synergistic degradation of proteinaceous materials. Low-temperature extrusion multi-enzyme synergistic degradation of proteinaceous materials is just low-temperature extrusion of proteinaceous materials added with a number of enzyme preparations (such as alkaline protease, complex protease, cellulase) to convert proteins in the materials into polypeptides. Included are a method for preparing protein and polypeptide by low-temperature extrusion multi-enzyme synergistic degradation of protein, and a method of preparing a powdery protein or polypeptide product through carrying out conventional extraction→separation (single enzyme and multi-enzyme synergistic hydrolysis or alkali-solution and acid-isolation)→purification→ultrafiltration→concentration→spray drying or freeze drying on pulverized substances obtained after low-temperature drying of extrudates of raw proteinaceous materials treated by low-temperature extrusion multi-enzyme synergistic degradation for preparing protein and polypeptide. The present invention is used for solving the problems of long process time, low protein extraction rate, low conversion rate of protein into polypeptide, high production cost, etc. for preparing proteins and polypeptides in the prior art.

It can be seen that for proteinaceous materials, when enzyme preparations such as alkaline protease, acid protease, neutral protease, complex protease, etc. are added, their optimum pH values are respectively different, and the types and characteristics of the enzymatically hydrolyzed proteins are different. During the extrusion process, the simultaneous addition of a number of enzyme preparations can overcome the limitations of enzymolysis of proteins by the addition of only one enzyme preparation. The process time required for the preparation of protein and polypeptide is less than the time required for the conventional process; the protein extraction rate is ≥80%; the conversion rate of protein into polypeptide is greater than 80%; and the peptide fragments having the molecular weight of less than 1000 Da of the polypeptide product is ≥75%.

In a further embodiment, the present invention relates to the addition of a number of enzyme preparations for the preparation of a raw material for protein and polypeptide before extrusion processing when preparing corn polypeptide powder and soy isolate protein powder, the enzyme preparation including: cellulase, hemicellulase, alkaline protease, neutral protease, acid protease, complex protease, etc. This can overcome the deficiencies brought by the addition of only one enzyme preparation, such as neutral protease or alkaline protease or acid protease, saying, the degradation of protein and the degree of protein's conversion into polypeptide are limited. The present invention can shorten the time required for the process of preparing proteins and polypeptides, increase the rate of protein extraction and the conversion rate of protein into polypeptide, shorten the time required to convert a protein material into a polypeptide material to ⅗-¾ of the time required for the conventional process, and enable the peptide fragments having a molecular weight of less than 1000 Da of the polypeptide product ≥75%.

In a further embodiment, the present invention relates to the low-temperature extrusion multi-enzyme synergistic degradation processing of vegetable fiber materials of stem, leaf, pod, root, fruit and the like from one or more of corn, wheat, barley, rice, sorghum, rape, soybean plants, which can increase the reduction of cellulose, hemicellulose and lignin in vegetable fiber materials by 3%-50%, making the extrudate more favorable for application in the fields like feed processing, papermaking, ethanol production by fermentation, production of xylan and xylitol, production of xylooligosaccharide, and production of foods and medicines for diabetics.

In a further embodiment, the present invention relates to low-temperature extrusion-multi-enzyme synergistic degradation processing of oily materials like one or more oily materials of soybean, corn germ, peanut, sunflower seed, rapeseed, sesame seed, yellow mustard seed, cottonseed, flax seed, castor seed, and perilla seed before solvent extraction, which can accelerate the destruction of the cell walls of oily materials, so that the oil in the cells rapidly accumulates into oil droplets, and this can accelerate the entry of the solvent into the cells and the extraction of the oil from the cells, thus reducing the residual oil rate to 0.3%-1.0%.

The present invention has the following advantages over the prior art (the extrusion-enzymolysis technique with the addition of only one enzyme preparation and the low-temperature extrusion technique):

increasing the yield of converting starch in starchy materials into glucose and maltose by 0.5%-3%;

reducing the meal residual oil rate after oil production by solvent extraction of oil materials to 0.3%-1.0%;

increasing the soluble substances of vegetable fiber materials by 3%-30%;

shortening the time required to convert a protein material into a polypeptide material to ⅗-¾ of the time required for the conventional process, and enabling the peptide fragments having a molecular weight of less than 1000 Da of the polypeptide product 75%.

### EXAMPLES

The following examples are intended to illustrate the present invention in more details, but are not to be construed as limitations to the scope of the present invention.

### Example 1

A process for producing glucose syrup using the extrudate from low-temperature extrusion-multi-enzyme synergistic degradation of dry-process degermed corn.

The method for producing glucose syrup by the technology of the present patent is as follows:

First, the raw corn was degermed by a dry process to obtain dry-process degermed corn flour, which was adjusted to have a moisture content ≤70%. At the same time, high-temperature-resistant α-amylase, glucoamylase and pullulanase were added; the extrusion barrel temperature was adjusted ≤80° C., and then, the material to be extruded was fed into a screw extrusion device for low-temperature extrusion processing. The extrudate was mixed with process water for producing syrup. The pH was adjusted, and high-temperature-resistant α-amylase or mesophilic α-amylase was added. The temperature was raised to 85° C.-95° C. and maintained for 1 min-20 min, and was then decreased to 55° C.-65° C. The above liquefied liquid was poured into a saccharification tank, followed by the pH adjustment and the addition of amyloglucosidase, for saccharification for 15 h. The resultant syrup had a DE value of about 100% and a glucose content DX of about 98%.

### Example 2

A process for producing oil by the extrudate from the low-temperature extrusion-multi-enzyme synergistic degradation of cell walls in soybean by solvent extraction.

First, soybean was pulverized to obtain soybean flour, which was adjusted to have a moisture content ≤70%. At the same time, cellulase, hemicellulase and pectinase were added; the extrusion barrel temperature was adjusted ≤80° C., and then, the material to be extruded was fed into a screw extrusion device for low-temperature extrusion processing. The extrudate was dried until the moisture content was ≤10%, and the dried soybean extrudate was introduced into a extractor to produce oil by solvent extraction, giving soybean meal and soybean oil. The residual oil rate of soybean meal was 0.3%-1.0%.

### Example 3

A process for low-temperature extrusion-multi-enzyme synergistic degradation of vegetable fibers in stem, leaf, root and fruit of corn.

First, stem, leaf, root and fruit of corn were pulverized to obtain a pulverized material which was adjusted to have a moisture content ≤70%. At the same time, cellulase, hemicellulase, pectinase and xylanase were added; the extrusion barrel temperature was adjusted ≤80° C., and then, the material to be extruded was fed into a screw extrusion device for low-temperature extrusion processing. The extrudate was mixed with water for saccharification treatment to obtain a glucose liquid. Soluble substances were increased by 3%-30%. The extrudate can be used to produce feed, alcohol, sugar products and their derivatives.

### Example 4

A process for producing fuel alcohol by the extrudate from low-temperature extrusion-multi-enzyme synergistic degradation of starch in dry-process degermed corn.

First, the raw corn was degermed by a dry process to obtain dry-process degermed corn flour, which was adjusted to have a moisture content ≤70%. At the same time, high-temperature-resistant α-amylase, glucoamylase, fungal α-amylase and protease were added; the extrusion barrel temperature was adjusted ≤80° C., and then, the material to be extruded was fed into a screw extrusion device for low-temperature extrusion processing. The extrudate was mixed with process water. The pH was adjusted, and the liquifying enzyme was added. The temperature was raised to 85° C.-95° C. and maintained for 1 min-15 min, and was then decreased to 55° C.-65° C. The pH was adjusted, and then alcohol-specific complex amyloglucosidase was added for saccharification. The temperature was maintained for 5 min-60 min, and was then decreased to 30° C.-40° C. The pH was adjusted to 3.5-5.5. Fuel alcohol protease and yeast were added to carry out fermentation. After a few hours, the desired alcohol content and starch liquor yield were achieved. Distillation was carried out to obtain alcohol. The technology of this project has the fermentation time of about 45 h-50 h and the alcohol content of 13%, while the prior art requires fermentation time of about 70 h to obtain the alcohol content of 13%.

### Example 5

A process for producing maltose syrup by the extrudate from low-temperature extrusion-multi-enzyme synergistic degradation of dry-process degermed corn.

First, the raw corn was degermed by a dry process to obtain dry-process degermed corn flour, which was adjusted to have a moisture content ≤70%. At the same time, high-temperature-resistant α-amylase, β-amylase and pullulanase were added; the extrusion barrel temperature was adjusted ≤80° C., and then, the material to be extruded was fed into a screw extrusion device for low-temperature extrusion processing. The extrudate was mixed with process water for producing syrup. The pH was adjusted, and high-temperature-resistant α-amylase or mesophilic α-amylase was added. The temperature was raised to 85° C.-95° C. and maintained for 1 min-20 min, and was then decreased to 55° C.-65° C. The above liquefied liquid was poured into a saccharification tank, followed by the pH adjustment and the addition of amyloglucosidase and maltotriohydrolase, for saccharification ≥40 h, giving the maltose content of about ≤103%.

### Example 6

A process for preparing corn polypeptide by low-temperature extrusion-multi-enzyme synergistic degradation.

A process for low-temperature extrusion-multi-enzyme synergistic degradation of protein of corn protein powder, wherein the moisture content of the corn protein powder before extrusion was less than 14%, and the powder should have the particle size to pass through an 80-mesh sieve. Then, an appropriate amount of water was added, with the moisture content adjusted to ≤70%. At the same time, hemicellulase, neutral protease and pectinase were added in an amount of 0.1 L-5 L or 0.1 kg-5 kg per one ton of raw material for preparing corn polypeptide before extrusion. The pH value of the added water was adjusted to 6.5-8.0. A commercially available single-screw (or twin-screw) extrusion device was used for extrusion processing of the above-mentioned materials. The extrusion barrel temperature was adjusted ≤80° C. The extrudate of the corn protein powder added with enzyme preparations for the preparation of corn polypeptide was subject to low-temperature drying at a drying temperature ≤70° C., with the moisture content ≤15% after drying, and then was pulverized to completely pass through a sieve having the pore diameter of 1.1-2.0 mm for use. Then, conventional extraction was performed to remove soluble non-protein substances (such as saccharides, lipids, etc.), followed by separation (single enzyme and multi-enzyme synergistic hydrolysis or alkali extraction and acid precipitation), purification, ultrafiltration, concentration, spray drying or freeze drying, to produce a powdered corn polypeptide product.

### Example 7

A process for preparing soy isolate protein powder by low-temperature extrusion-multi-enzyme synergistic degradation.

A process for preparing soy isolate protein powder by low-temperature extrusion-multi-enzyme synergistic degradation of defatted soybean, wherein the defatted soybean meal after the solvent extraction of oil had a moisture content of less than 14% before extrusion, and the powder should have the particle size to pass through an 80-mesh sieve. Then, an appropriate amount of water was added, with the moisture content adjusted to ≤70%. At the same time, alkaline protease, hemicellulose and pectinase were added in an appropriate amount of 0.1 L-6 L or 0.1 kg-6 kg per one ton of defatted soybean meal. The pH value of the added water was adjusted to 8-12. A commercially available single-screw (or twin-screw) extrusion device was used. The extrusion barrel temperature was adjusted ≤80° C. The extrudate was subject to low-temperature drying at a drying temperature ≤70° C., with the moisture content ≤15% after drying, and then was pulverized to completely pass through a sieve having the pore diameter of 1.1-2.0 mm for use. The pulverized material of the above extrudate after low-temperature drying was then subject to conventional extraction to remove soluble non-protein substances (such as sacchrides, lipids, etc.), and to separation (single enzyme and multi-enzyme synergistic hydrolysis or alkali extraction and acid precipitation), concentration and spray drying to form a powdered soy isolate protein product.

The present invention is not limited by the specific embodiments described herein in scope. The foregoing written description is considered to be sufficient to enable those skilled in the art to practice the present invention. In addition to those shown and described herein, various modifications of the present invention will be understood by those skilled in the art from the foregoing description, and the modifications are intended to fall within the scope of the appended claims.

1. A material pretreatment method by low-temperature extrusion-multi-enzyme synergistic degradation, including preparing the material to be extruded added with enzyme preparations and having a certain moisture content before extrusion, wherein two or more enzyme preparations are added to the material before extrusion, and then, the material to be extruded is fed into a screw extrusion device for low-temperature extrusion processing to form an extrudate.

2. (canceled)

3. A material pretreatment method by low-temperature extrusion-multi-enzyme synergistic degradation, including preparing the material to be extruded added with enzyme preparations and having a certain moisture content before extrusion, wherein the added enzyme preparations include: two or more of added high-temperature-resistant α-amylase, highly-efficient thermo stable α-amylase, mesophilic α-amylase, amyloglucosidase, glucoamylase, highly-efficient amyloglucosidase, potent amyloglucosidase, protease, complex enzyme, and two or more of added alkaline protease, neutral protease, acid protease, complex protease and papain; characterized in that the added enzyme preparations further include: two or more of complex amyloglucosidase, pullulanase, maltobiohydrolase, maltotriohydrolase, cellulase, hemicellulase, pectinase, xylanase, β-amylase, fungal α-amylase, fuel alcohol protease, alcohol-specific complex amyloglucosidase, and then, the material to be extruded is fed into a screw extrusion device for low-temperature extrusion processing to form an extrudate.

4. The material pretreatment method by low-temperature extrusion-multi-enzyme synergistic degradation according to claim 1, including preparing the material to be extruded added with enzyme preparations and having a certain moisture content before extrusion, the material to be extruded including: one or more of rice, degermed corn, corn with germ, sorghum rice, barley, wheat and their starch, and crude starch added with enzyme preparations, and one or more of cassava whole flour, potato whole flour and their starch, and crude starch added with enzyme preparations, characterized in that the material to be extruded further including: one or more starchy materials of sweet potato whole flour, millet, black kerneled rice, buckwheat, oat, rye and their starch, and crude starch added with enzyme preparations.

5. The material pretreatment method by low-temperature extrusion-multi-enzyme synergistic degradation according to claim 1, including preparing the material to be extruded added with enzyme preparations and having a certain moisture content before extrusion, characterized in that the material to be extruded including: one or more oily materials of soybean, corn germ, peanut, sunflower seed, rapeseed, sesame seed, yellow mustard seed, cottonseed, flax seed, castor seed, and perilla seed added with enzyme preparations.

6. The material pretreatment method by low-temperature extrusion-multi-enzyme synergistic degradation according to claim 1, including preparing the material to be extruded added with enzyme preparations and having a certain moisture content before extrusion, characterized in that the material to be extruded including: stem, leaf, pod, root, fruit vegetable fiber materials from one or more of corn, wheat, barley, rice, sorghum, rape, soybean plants added with enzyme preparations.

7. The material pretreatment method by low-temperature extrusion-multi-enzyme synergistic degradation according to claim 1, including preparing the material to be extruded added with enzyme preparations and having a certain moisture content before extrusion, characterized in that the material to be extruded including: one or more materials in the filtered mash residues from brewing beer with rice, degermed corn, sorghum rice, barley and wheat as auxiliary materials, one or more materials in the filtered residues of sugar liquid from producing syrup with rice, degermed corn, sorghum rice, barley and wheat as raw materials, one or more materials in the dregs or filtered residues from producing alcohol with rice, degermed corn, corn with germ, sorghum rice, barley and wheat as raw materials, one or more materials in the defatted cakes from producing oil with soy, corn germ, peanut, sunflower seed, rapeseed, sesame, yellow mustard, rice bran and cottonseed as raw materials, and one or more proteinaceous materials in soy protein powder, corn protein powder, barley protein powder, wheat protein powder, peanut protein powder, sunflower seed protein powder, rapeseed protein powder, rice bran protein powder, sesame protein powder and cottonseed protein powder.

8. (canceled)

9. The material pretreatment method by low-temperature extrusion-multi-enzyme synergistic degradation according to claim 1, including preparing the material to be extruded added with two or more enzyme preparations and having a certain moisture content before extrusion, and then, feeding the material to be extruded into a screw extrusion device for low-temperature extrusion processing, characterized in that the screw extrusion device comprises one or more of a single-screw extruder, a twin-screw extruder, and a tri-screw extruder.

10. (canceled)

11. The material pretreatment method by low-temperature extrusion-multi-enzyme synergistic degradation according to claim 2, including preparing the material to be extruded added with enzyme preparations and having a certain moisture content before extrusion, the material to be extruded including: one or more of rice, degermed corn, corn with germ, sorghum rice, barley, wheat and their starch, and crude starch added with enzyme preparations, and one or more of cassava whole flour, potato whole flour and their starch, and crude starch added with enzyme preparations, characterized in that the material to be extruded further including: one or more starchy materials of sweet potato whole flour, millet, black kerneled rice, buckwheat, oat, rye and their starch, and crude starch added with enzyme preparations.

12. The material pretreatment method by low-temperature extrusion-multi-enzyme synergistic degradation according to claim 2, including preparing the material to be extruded added with enzyme preparations and having a certain moisture content before extrusion, characterized in that the material to be extruded including: one or more oily materials of soybean, corn germ, peanut, sunflower seed, rapeseed, sesame seed, yellow mustard seed, cottonseed, flax seed, castor seed, and perilla seed added with enzyme preparations.

13. The material pretreatment method by low-temperature extrusion-multi-enzyme synergistic degradation according to claim 2, including preparing the material to be extruded added with enzyme preparations and having a certain moisture content before extrusion, characterized in that the material to be extruded including: stem, leaf, pod, root, fruit vegetable fiber materials from one or more of corn, wheat, barley, rice, sorghum, rape, soybean plants added with enzyme preparations.

14. The material pretreatment method by low-temperature extrusion-multi-enzyme synergistic degradation according to claim 2, including preparing the material to be extruded added with enzyme preparations and having a certain moisture content before extrusion, characterized in that the material to be extruded including: one or more materials in the filtered mash residues from brewing beer with rice, degermed corn, sorghum rice, barley and wheat as auxiliary materials, one or more materials in the filtered residues of sugar liquid from producing syrup with rice, degermed corn, sorghum rice, barley and wheat as raw materials, one or more materials in the dregs or filtered residues from producing alcohol with rice, degermed corn, corn with germ, sorghum rice, barley and wheat as raw materials, one or more materials in the defatted cakes from producing oil with soy, corn germ, peanut, sunflower seed, rapeseed, sesame, yellow mustard, rice bran and cottonseed as raw materials, and one or more proteinaceous materials in soy protein powder, corn protein powder, barley protein powder, wheat protein powder, peanut protein powder, sunflower seed protein powder, rapeseed protein powder, rice bran protein powder, sesame protein powder and cottonseed protein powder.

15. The material pretreatment method by low-temperature extrusion-multi-enzyme synergistic degradation according to claim 2, including preparing the material to be extruded added with two or more enzyme preparations and having a certain moisture content before extrusion, and then, feeding the material to be extruded into a screw extrusion device for low-temperature extrusion processing, characterized in that the screw extrusion device comprises one or more of a single-screw extruder, a twin-screw extruder, and a tri-screw extruder.
